(19) United States
(12) Patent Application Publication
(10) Pub. No.: US 2006/0229580 A1
(43) Pub. Date: Oct. 12, 2006

(54) ABSORBENT ARTICLES

(75) Inventors: Maria Raidel, Nurnberg, GE (GE); Franz Aschenbrenner, Kastl, DE (DE)

Correspondence Address: SENNIGER POWERS, ONE METROPOLITAN SQUARE, 16TH FLOOR, ST LOUIS, MO, 63102, US

(73) Assignee: HAKLE-KIMBERLY DEUTSCHLAND GMBH, Munchen, DE (DE)

(21) Appl. No.: 11/423,065

(22) Filed: Jun. 8, 2006

Related U.S. Application Data
(62) Division of application No. 10/204,333, filed on Dec. 10, 2002.

(30) Foreign Application Priority Data
Feb. 18, 2000 (DE) 100 07 566.5
Dec. 29, 2000 (DE) 100 65 680.3

(51) Int. Cl. A61F 13/15

(52) U.S. Cl. 604/374

(57) ABSTRACT

The present invention concerns absorbent articles. This invention concerns in particular an absorbent article comprising cellulose fibers (14) present at least partially in the form of granules. Furthermore, the present invention also concerns the use of cellulose fibers (14) present at least partially in the form of granules as a material in an absorbent article.

The present invention concerns absorbent articles.

Absorbent articles for a single use have been known for many years. They are used, for example, as sanitary napkins, panty liners, diapers or incontinence pads. These disposable absorbent articles have in common the fact that they have a layer that holds liquid. The liquid-storing layer may be made, for example, of cellulose.

One disadvantage of the known disposable absorbent articles has proven to be the fact that the conventional pure cellulose layers have a limited retention capacity as a liquid-retaining material. Furthermore, the retention capacity of a cellulose material after being exposed to fluids is not very great, especially under pressure. Finally, deformed cellulose material retains any shape it has assumed, which is often perceived as unpleasant by the person wearing the absorbent article. When a wearer is mentioned below, this should be understood to refer to both male and female wearers.

In addition, there are known absorbent articles for a single use which contain superabsorbent materials as the liquid-storing layer. Superabsorbent materials are capable of absorbing a liquid weight many times greater than their dry weight and retaining it to a certain extent even when pressure is applied. Superabsorbent materials are disclosed, for example, in EP-A-0 339461.

One problem with using superabsorbent materials in the liquid-retaining layer of absorbent articles is that superabsorbent materials undergo an increase in volume when they absorb liquid, i.e., they “swell” up. This results in the absorbent article becoming “full” with a decline in comfort for the wearer. The individual components of the superabsorbent material also tend to stick together when exposed to a liquid, which leads to a great reduction in the theoretical liquid absorption power (so-called “gel blocking”). Gel blocking causes a restricted distribution of the liquid that penetrates into the absorbent article. When exposed to a large amount of liquid, this can result in the liquid no longer being absorbed completely in the absorbent article, although theoretically sufficient storage capacity should still be available, in which case the wearer of the article would perceive a feeling of wetness or uncleanliness on the skin and there would also be the risk of soiling the wearer's clothing. Finally, the absorbent article may also undergo permanent deformation due to the gel blocking effect, further reducing the comfort in wearing the article.

Furthermore, it would be desirable in the respective technical field to create absorbent articles with such materials that would not pose any health risk for the user of the absorbent article. In addition, such materials should preferably be produced biologically and should especially preferably be biodegradable, because the ultimate disposal of disposable products is still an environmental problem.

It is thus the object of the present invention to provide absorbent articles which do not have the disadvantages of known products described above. This object is achieved by the present invention with the absorbent articles stated in independent Patent claim 1. Additional advantageous embodiments, details and aspects of the present invention are derived from the dependent patent claims, the description and the drawings.

Another object of the present invention is to provide materials whose use will have a positive effect on the flow properties and retention capacity of an absorbent article. Use of such materials is claimed in independent Patent claim 52.

An additional object of the invention is to provide absorbent articles for the field of female hygiene which are to be worn in a particularly discrete manner. This object is solved by independent claim 55. Preferred developments thereof are explained more closely in the respective subclaims.

Before the present invention is described in detail, some terms should first be defined to facilitate an understanding of the object according to this invention:

Absorbent article: The term “absorbent article”, as used in the present patent application, is understood to refer to articles suitable for absorption, in particular for absorption of body fluids. This includes in particular the absorption of urine, blood and fecal matter. Absorbent articles, according to the present invention, are often disposable articles, but they need not necessarily be disposable. Examples of absorbent articles according to the present invention include sanitary pads, in particular sanitary napkins and panty liners, diapers, incontinence pads, bandages and similar articles.

Liquid-permeable cover layer: This is understood to refer to the “top” layer of an absorbent article which faces the body when the absorbent article is in use and is essentially in contact with the wearer's body. This cover layer is essentially liquid permeable in the sense that such body fluids as blood or urine can penetrate through the cover layer into the other areas of the absorbent article.

Liquid-impermeable backing layer: This is understood to refer to the “bottom” layer of an absorbent article which is arranged on the side facing away from the wearer's body during use. This layer is essentially liquid-impermeable in the sense that the body fluids discharged from the body which have penetrated into the absorbent article, as far as this layer, cannot spread further in a direction away from the wearer's body. Thus, soiling of clothing in which the absorbent article is arranged is prevented. Although the backing layer is in many cases the layer facing away from the wearer's body and the farthest away from it, there may also be other layers even farther away from the wearer's body such as adhesive layers and nonwovens.

Absorbent body: An absorbent body according to the present invention comprises any absorbent material which has been rendered in any desired form. Absorbent bodies may be arranged centrally in an absorbent article, or they may cover the entire length and width of the absorbent article. Other arrangements are also comprised. In the typical case, an absorbent body is arranged between a liquid-permeable cover layer and a liquid-impermeable backing layer, although this need not necessarily be the case.

Fine cellulose fibers: Cellulose fibers are fibers obtained from cellulose by methods with which those skilled in the art are familiar. Such cellulose fibers are regarded as “fine” if they conform to the diameter and length or thickness specifications given in the accompanying claims and in the description.

Sorted: The granulated cellulose fibers are sorted by sifting the grains obtained in the process and checking them for suitability.

Free-flowing: A material is understood to be free-flowing if it remains free-flowing not only when dry but also after being exposed to liquid.

Superabsorbent material: This term refers to a water-swellable, essentially water-insoluble material that is capable of absorbing at least approximately 10 times its weight in water, preferably approximately 20 times its weight and especially approximately 50 times its weight or more. The superabsorbent material may be formed from an organic material, comprising natural materials such as agar, pectin and guar gum as well as synthetic materials such as synthetic hydrogel polymers. Synthetic hydrogel polymers comprise, for example, carboxymethylcellulose, alkali metal salts of polyacrylic acid, polyacrylamides, polyvinyl alcohol, ethylene-maleic anhydride copolymers, polyvinyl ether, hydroxypropylcellulose, polyvinylmorpholinone, polymers and copolymers of vinylsulfonic acid, polyacrylates, polyacrylamides, polyvinylpyridine and the like. Other suitable polymers include hydrolyzed acrylonitrile-grafted starch, acrylic acid-grafted starch and isobutylene-maleic anhydride copolymers and mixtures thereof. The hydrogel polymers are preferably slightly crosslinked to make the materials essentially water-insoluble. The crosslinking may be induced, for example, by means of radiation or by covalent, ionic, van der Waals or hydrogen bonding. Suitable materials are available from various commercial retailers such as Dow Chemical Company, Hoechst Celanese Corp., BASF and Stockhausen Inc. A superabsorbent material may be in the form of particles, beads, flocks, fibers, rods, films or any number of other geometric shapes. If it is in the form of particles or beads, it may be desirable for the particles or beads to have a maximum cross-sectional dimension of approximately 20 micrometers to approximately 2000 micrometers, preferably approximately 60 micrometers to approximately 1000 micrometers.

Water retention capacity: The water retention capacity according to the present invention describes a property of a material according to the method described on pages 31 to 32 of the present patent application.

All other technical terms used in the present patent application should be understood according to the definitions in Rbmpp Chemielexicon [Römpp's Chemical Dictionary], Thieme-Verlag, 9^{th} edition, 1995, unless otherwise defined here.

According to a first aspect, the present invention concerns an absorbent article comprising cellulose fibers at least partially in the form of granules. An absorbent article comprising cellulose fibers at least partially in the form of granules has the advantage that optimal adaptation of the absorbent article to the individual body shape of the wearer can be achieved, which greatly increases wearing comfort. This advantage is achieved because the material comprising cellulose fibers at least partially in the form of granules is free-flowing and remains free-flowing even after being exposed to fluid.

This also achieves the result that the functionality of the article is maintained even in a deformed condition.

The cellulose fibers, at least partially in the form of granules, may be obtained from wood and/or other plant fibers. Cellulose fibers obtained from wood and at least partially in the form of granules are preferred. Other suitable plant fibers comprise, for example, apple fibers, orange fibers and wheat fibers. Those skilled in the art will be familiar with methods of producing cellulose fibers from wood and/or other plant fibers so that they are at least partially in the form of granules.

According to a preferred embodiment of the present invention, the absorbent article also comprises at least the following components:
a liquid-permeable cover layer facing the wearer's body when the absorbent article is in use,
a liquid-impermeable backing layer facing away from the wearer's body when the absorbent article is in use,
an absorbent body arranged between the liquid-permeable cover layer and the liquid-impermeable backing layer, with the cellulose fibers at least partially in the form of granules being arranged in the absorbent body.

In addition, wings or other lateral projections may also be provided to simplify the attachment of the absorbent article to an item of clothing for use.

It is especially preferable for the absorbent body to be arranged centrally in the absorbent article with a distance from the side edges of the absorbent article in each direction.

A special embodiment of the absorbent article according to the first aspect of the present invention is an embodiment where the liquid-permeable top cover layer has folds running in a longitudinal direction. These folds are designed so that the liquid-permeable layer partially covers the absorbent body even on the side facing away from the wearer's body when wearing the absorbent article. This is achieved first by the fact that the absorbent body is not bonded to the layer beneath it on its entire “underside” but instead is bonded to it only in a narrow central zone. Second, two folds are incorporated into the liquid-permeable layer, also wrapping around the absorbent body in the longitudinal direction of the absorbent article and at least partially on the underside. This design yields the result that despite being embedded between the liquid-impermeable backing layer and the liquid-permeable cover layer, the absorbent body still remains flexible and can be adapted very well to the anatomical features of the wearer's body.

If the liquid-permeable layer and the liquid-impermeable layer are bonded to one another in their edge areas in such a way as to form a tightly sealed interior space, the absorbent body may be formed by a material loosely, displaceably put against each other, which remains free-flowing even after coming in contact with fluid, so that the material can move freely in the entire interior space.

According to another aspect, the present invention concerns an absorbent article having a liquid-impermeable layer facing away from the wearer's body during use of the article and an absorbent body enclosed by a liquid-permeable sheathing and including an absorbent material which remains free-flowing even after coming in contact with a fluid, where the absorbent body is bonded to the liquid-impermeable layer in a central area thereof. It is not necessary here for the absorbent body and the liquid-impermeable layer to be in direct contact. If one or more other layers are arranged on the liquid-impermeable layer, the absorbent body is attached to the top side of the top layer of these layers.

The absorbent material in the absorbent body preferably remains free-flowing even while being worn and after coming in contact with fluid. It is especially preferable for the material to remain free-flowing up to a liquid load of at least 10 ml/g material. This makes it possible for the absorbent article to conform to the individual wearer's body during different movements and different types of stresses. In other words, the absorbent body “flows” and when there is a lateral load or a lateral pressure due to the wearer's thigh, it can yield slightly to this pressure or load by displacing absorbent material into less stressed areas. When the load or the pressure is removed, displaced particles can flow back to the starting position where they are again available to absorb liquid. On the other hand, particles may also be repositioned by these movements, and thus previously unused absorbent capacity and storage capacity can be utilized in this way. When an “absorbent body” is mentioned in the present case, it is also understood to refer to a “storage body.”

The granules according to this invention have a structure like a cotton ball. Thus, they have a certain void volume, i.e., hollow volume leading to a large surface area. This special design of the granules presumably leads to a greater mobility and free-flowing property, but it also ensures voids in which liquids, in particular blood cells, for example, can be collected and stored.

Due to the absorbent body which also serves as a storage layer at the same time and includes the absorbent material according to this invention which remains free-flowing even after coming in contact with a liquid, the following advantageous properties are obtained for the absorbent articles according to this invention:
rapid liquid uptake (good penetration into the free-flowing material and good wetting of the material),
good retention of the liquid (locking the liquid in, even under a pressure load),
good absorbent capacity (absorption with practically no increase in volume),
preventing lumps when coming in contact with a fluid,
best possible adaptation to the individual wearer's body,
great softness of the article combined with good comfort in wearing,
very good liquid transport and good liquid distribution,
no collapsing or “bottoming out” as is known to occur with other cellulose absorbent bodies,
healthy absorbent material,
biologically producible and usable absorbent material, and
optimum utilization of the available absorbent material.

Especially with the absorbent article according to the above-mentioned additional aspect of this invention, an optimum adaptation to the anatomical features of the wearer's body is obtained when wearing this absorbent article because the sheathed absorbent body is practically “exposed,” i.e., it is not separated from the wearer's body by a liquid-permeable layer covering the entire article, and it extends vertically, i.e., the third dimension, namely toward the wearer's body. The fluid excreted from the wearer's body can be absorbed into the absorbent body directly at the point of exit from the wearer's body and is then conveyed further or stored there.

The bond between the liquid-impermeable backing layer and the absorbent body enclosed by a liquid-permeable layer may be accomplished in any suitable manner. For example, a bond produced by an adhesive has proven advantageous in machine production of the absorbent article according to this invention. However, the backing layer and the absorbent body may also be firmly joined together by sewing, for example, in which case it is of course important to be sure that the liquid-impermeable backing layer is not damaged so that fluid can pass through it.

In addition, it has proven to be advantageous if the absorbent article according to this invention has another layer of soft material which can serve as the secondary reservoir on the side of the liquid-impermeable layer facing the wearer's body. This additional layer further increases the wearer's comfort in wearing the absorbent article. This additional layer can also store liquid not yet absorbed by the main absorbent body, but of course the absolute storage capacity of the additional layer is much lower in comparison with that of the actual absorbent body. Suitable materials for the additional layer comprise coform (polypropylene-cellulose mixtures), airlaid materials (synthetic fiber-cellulose mixtures) and nonwoven materials such as spunbonded nonwovens and carded nonwovens.

According to another aspect of the present invention, the absorbent material which remains free-flowing, even after coming in contact with a liquid, is embedded in a matrix of fiber material. The material can be incorporated homogeneously into the fiber matrix, so that the components of the material which remains free-flowing even after coming in contact with a liquid are distributed uniformly over the fiber structure and incorporated into it. As an alternative, however, the absorbent body may also have a layered structure, where the absorbent material which remains free-flowing even after coming in contact with a liquid is sandwiched between two or more layers of fiber material. Finally, with the sandwich structure described last, additional absorbent material which still remains free-flowing even after coming in contact with a liquid may also be incorporated into the fiber layers. Especially suitable materials for use as the fiber materials for the above-mentioned purposes comprise cellulose or a mixture of cellulose and polypropylene, i.e., a so-called coform material. An even more optimal distribution of liquid in the absorbent article according to this invention is achieved through the use of this fiber material because the fibers have a certain absorbency and can transport the liquid directionally. The ratio of absorbent material which remains free-flowing even after coming in contact with a liquid and fiber material is preferably 1 to 25 wt % to 99 to 75 wt % and in particular 10 to 15 wt % to 90 to 85 wt %.

According to another aspect of the present invention, the absorbent body may also include at least one care substance in an absorptive bond in addition to the absorbent material which remains free-flowing even after coming in contact with a liquid. This refers primarily to substances which protect the skin of the wearer of the absorbent article according to this invention. Suitable substances include, for example, extracts of aloe vera, calendula and/or chamomile.

It is especially advantageous if the care substances are enclosed in microcapsules. The microcapsules may be mixed with the absorbent material which remains free-flowing even after coming in contact with a liquid. The enclosures of the microcapsules should be designed so that they rupture when the absorbent article according to this invention is worn and thus release the substance or substances. The microcapsules may rupture under pressure, heat and/or friction, for example. Microencapsulation of substances has long been known for use in the printing industry, for example.

The cellulose fibers according to the present invention which are at least partially in the form of granules may be completely in the form of granules, essentially in the form of granules or a mixture of fibers and granules. Both the granules and the mixture are free-flowing, which is associated with the advantages described above.

According to a preferred embodiment of the present invention, the particles in the granules have a diameter of 50 to 3000 μm. It is especially preferable for them to have a diameter of 400 μm.

An average powder density of the granules (measured according to DIN 53,468) of 30 to 600 g/l is also preferred. An average powder density of 100-300 g/l±15% is especially preferred here.

For example, the granules may be granules of the product LC 200, cellulose fibers from the company J. Rettenmaier+Söhne GmbH+Co. [J. Rettenmaier and Sons Inc.], Germany.

If fibers are also present in the cellulose fibers of this invention which are at least partially in the form of granules, these fibers will preferably have an average fiber length of 100 to 600 μm. An average fiber length of 300 μm is especially preferred. According to a preferred embodiment of the present invention, the average fiber thickness of the fibers is 10 to 50. An average fiber thickness of 20 μm is especially preferred.

According to a preferred embodiment of the present invention, fibers having the properties specified above are used to produce the granules.

If the cellulose fibers according to this invention which are at least partially in the form of granules are in the form of a mixture of fibers and granules, this may be a “true” mixture, i.e., the cellulose fibers and the resulting granules are brought in contact and mixed by methods with which those skilled in the art are familiar. On the other hand, the mixture may also consist of particles which are in the form of granules, but fibers are present on the surface of the individual granule particles. Such “mixtures” can be produced by suppressing the process of winding fine cellulose fibers or granulating them to granules so promptly that individual unwound fibers or incompletely granulated fibers project out of the granules.

The mixture of granules and fibers preferably includes from 10 wt % to 80 wt % fibers. It is especially preferred for the mixture to contain 60-70 wt % granules and 30 to 40 wt % fibers.

In addition to the cellulose fibers which are at least partially in the form of granules, the absorbent body of the absorbent article may also contain other materials. The material composition may be selected so that the above-mentioned functions are assumed by a material or distributed among different materials. These materials may comprise: superabsorbers, superabsorbent materials in particle form, superabsorbent fibers, zeolites, fibers of cellulose, viscose staple fiber or synthetic staple beads of different lengths, polystyrene, etc.

With regard to the properties of the cellulose fibers which are at least partially in the form of granules, it should also be pointed out that these particles have a structure suitable for suppressing odors. If the cellulose fibers according to this invention, which are at least partially in the form of granules, are used as the liquid absorbing or liquid storing material, it is possible to omit the use of zeolites, for example, to bind odors, which is another advantage of the absorbent articles according to this invention. The cellulose fibers according to the present invention which are at least partially in the form of granules also have advantages with regard to the amount of absorbent material to be used. In comparison with polyacrylates which are used as superabsorbers, the cost of the cellulose fibers according to this invention, which are at least partially in the form of granules, is approximately of the same order of magnitude as the cost of conventional cellulose.

As indicated above, the present invention also concerns preventing unpleasant odors in use of absorbent articles according to this invention. It is proposed according to this invention that bactericidal, fungicidal and/or virucidal substances be applied by immobilization or adsorptive binding in or on the absorbent material which remains free-flowing even after coming in contact with a liquid. If cellulose fibers of the invention which are at least partially in the form of granules are used as the absorbent material according to this invention, they will have the structure described above, i.e., the material also has a large internal surface area in addition to its external surface. It is important for the bactericidal, fungicidal and virucidal substances to be immobilized on the absorbent material, because if the substances are released, they could lead to skin irritation of the wearer of the absorbent articles. Mainly metabolic products of microorganisms are responsible for the unpleasant odors that occur when using absorbent articles; in other words, odors can be effectively suppressed or prevented by inhibiting the growth and/or reproduction of the microorganisms or by killing the microorganisms. With these substances it is possible to inhibit and suppress the growth of microorganisms, so that in addition to preventing odors effectively in the cellulose material which is at least partially in the form of granules according to this invention, odors can also be prevented by the additional use of the above-mentioned bactericidal, fungicidal and virucidal substances, which provides additional safety from adverse effects for the wearer of an absorbent article according to this invention.

Suitable bactericidal substances include, for example, chlorinated levulinic acid and alkyldimethylbenzylammonium halides.

In addition to the absorbent body which contains the components which remain free-flowing even after coming in contact with a liquid, the absorbent body may also have other areas. In one such case, the absorbent body portion containing the free-flowing material is designed preferably as at least one core piece whose length l is smaller than the length L of the absorbent article and whose width b is smaller than the width B of the absorbent article.

The absorbent body may be bonded to the underlying layer over the entire contact area, e.g., by an adhesive. Preferably only a part of the absorbent body is bonded to the underlying layer. A wide variety of different embodiments are conceivable here, e.g., attachment in strips by securing the absorbent body to the underlayer with strips of adhesive. Expressed in general terms, the area or area portion by which the absorbent body is bonded to the underlying layer is smaller than the area or area portion, l×b. The length λ of the absorbent body/underlying layer bond is less than or equal to the length l, and the width β of the absorbent body/underlying layer bond is less than or equal to the width b.

The absorbent body may consist of a compartment or be subdivided into multiple subcompartments, which are completely separated from one another or may communicate with one another, in which case the particles may also escape to an adjacent compartment when a pressure load is applied to the compartment.

With an absorbent body design having three compartments, for example, the central compartment, for example, may be filled with the cellulose fibers according to this invention which are at least partially in the form of granules or mixtures of cellulose fibers according to this invention which are at least partially in the form of granules and other additives such as superabsorbent materials. A suitable compartment division and arrangement is described, for example in WO 98/43684.

A superabsorbent material or other additives may be added to the absorbent material at any possible location and in any conceivable configuration. The superabsorbent material is preferably a polyacrylate. The superabsorbent material and the cellulose fibers which are at least partially in the form of granules as well as optionally the fiber material may be in the form of a mixture or they may be arranged in layers.

According to a preferred embodiment of the present invention, the fiber material is arranged between a layer of the superabsorbent material and a layer of the cellulose fibers which are at least partially in the form of granules, where the layer of superabsorbent material is especially preferably arranged on the side of the fiber layer facing away from the wearer's body (the “bottom”), and the layer of cellulose fibers which are at least partially in the form of granules is especially preferably arranged on the side of the fiber layer facing the wearer's body (the “top”).

According to an alternative embodiment of the present invention, the absorbent material and/or the cellulose fibers according to the present invention which are at least partially in the form of granules and/or the superabsorbent material are in the form of a mixture.

In order to improve the full efficiency of the cellulose fibers which are at least partially in the form of granules, it may be advantageous to coat these fibers with an agent that retards absorption of liquid. Such agents that retard liquid uptake are known in the industry. As an alternative, the cellulose fibers which are at least partially in the form of granules can be made hydrophobic by using suitable agents which are known by those skilled in the art.

The cellulose fibers which are at least partially in the form of granules advantageously have a water retention capacity of at least 6 g H₂O/g of cellulose material which is at least partially in the form of granules. Its water retention capacity is especially advantageously 8 to 16 g H₂O/g of the cellulose fibers which are at least partially in the form of granules.

The cellulose fibers according to this invention which are at least partially in the form of granules may be used as a material in an absorbent article. Because of the advantages described above, there are some advantages for an absorbent article including the cellulose fibers according to this invention which are at least partially in the form of granules.

The advantages mentioned above include in particular an improved fluidity, i.e., the property of being free-flowing, of the absorbent material in an absorbent article as described above. If improving the fluidity of an absorbent article is a special goal, then the cellulose fibers according to this invention which are at least partially in the form of granules are preferably used essentially in the form of granules. “Essentially” as used here means that of the cellulose fibers which are at least partially in the form of granules, more than 90% of the fibers are actually in granular form.

If rewetting, in particular rewetting under pressure, is to be improved in an absorbent article, this can be achieved in particular by having the cellulose fibers, which are at least partially in the form of granules, be essentially in fiber form. “Essentially” as used here means 30-50 wt % of the fibers by weight.

When using mixtures of, for example, superabsorbers and the cellulose fibers according to this invention, which are at least partially in the form of granules, it is important to be sure that these mixtures do not separate because this could result in not all the potentially absorbent material or potential material having storage capability being utilizable, i.e., it could lead to so-called dead zones.

Thus, the present invention makes available absorbent articles that adapt optimally to the contours of the wearer's body. These articles are also characterized in that they do not undergo any increase in volume when suitable absorbent materials come in contact with a liquid, i.e., they do not swell up. Finally, the absorbent articles according to this invention can also absorb liquid optimally even in the deformed condition.

The absorbent articles according to this invention also conform optimally to the contours of the wearer's body. These articles can thus be worn very close to the body (anatomical form fitting), which has the advantage that a liquid can be absorbed immediately after leaving the body, so there is no feeling of wetness on the skin of the wearer. The feeling of dryness on the skin of the wearer is also achieved by the fact that the absorbent material is arranged centrally in the absorbent article according to this invention. Finally, the design of the absorbent article according to this invention also allows, for example, the ends of a sanitary napkin to be kept extremely thin, which thus makes it possible for such an article to be worn very discretely.

It is also especially advantageous according to the present invention for the cellulose fiber material according to this invention, which is present at least partially in the form of granules, to be obtained from biological materials which are readily accessible and inexpensive. These materials are also completely safe from the standpoint of health. In addition, they have the advantage that they are biodegradable, which is very important for disposable articles in particular.

In addition to the element described above (=primary storage material) which is referred to as the absorbent body and also serves as a liquid storage layer, the absorbent article according to this invention may also have another storage layer (so-called secondary storage). This additional storage layer is preferably designed as a sheet between the absorbent body and the liquid-impermeable layer facing away from the wearer's body. This material forming the additional storage layer may also have an absorbent effect to achieve a better distribution of fluid. This additional storage layer is intended only for “emergencies” if the capacity limit of the absorbent body (primary storage layer) is exceeded for whatever reasons. Suitable materials for the additional storage layer (secondary storage layer) comprise, for example, coform materials, conventional cellulose, conventional cellulose fiber mixtures (air-laid), nonwovens or tissue wadding.

The absorbent articles according to this invention having the novel absorbent body may be used, for example, in the field of hygiene, e.g., as sanitary napkins in feminine hygiene, in particular ultrathin sanitary napkins, or panty liners. In addition, the absorbent article according to this invention may also be designed as disposable baby diapers or children's training pants or as incontinence liners or as a bandage in the field of medicine.

If the absorbent article has a liquid-permeable cover layer facing the wearer's body when in use, then another cover layer having a central opening (a so-called porthole) arranged above the absorbent body may also be arranged beneath this liquid-permeable layer. Corresponding absorbent articles are described, for example, in German Patent Application No. 196404517. This porthole design is especially suitable for feminine hygiene articles in particular.

The absorbent article stated in the aforementioned German patent application may have the following structure. A liquid-impermeable layer is provided on the side of the absorbent article facing away from the wearer's body during use. A primary storage layer is arranged above this liquid-impermeable layer. This is followed by a secondary storage layer above that. Above the secondary storage layer there is a compensation layer, and above the compensation layer there is a cover layer having a central opening. A corresponding cover layer provided with an opening may also be present in the absorbent articles according to the present invention. Finally, the absorbent article according to the present invention may also include an upper liquid-permeable layer facing the wearer's body during use of the absorbent article. The secondary storage layer may have at least one compressed area.

Conventional cellulose, for example, is a suitable material for the secondary storage layer. Local compression of the secondary storage layer may be produced, for example, by embossing grooves in the storage layer. The storage material located beneath the embossed grooves is then compressed, while the grooves contribute toward a directional distribution of fluid in the storage layer and in the absorbent article.

The cover layer having the central opening is made of a mixture of cellulose and polymerized alkene, for example. Suitable mixtures preferably contain at least 50 wt % polymerized alkene. Very good results are achieved when the amount of polymerized alkene amounts to 50 to 80 wt %, in particular 60 wt %. The cover layer may also be composed of two layers such that a first layer of a mixture of cellulose and polymerized alkene is applied to a second carrier layer of polymerized alkene, with the first layer of a mixture of cellulose and polymerized alkene being connected to the liquid-impermeable layer facing the wearer's body when the absorbent article is in use and the second carrier layer being connected to the compensation layer. Preferred polymerized alkenes include polyethylene, polypropylene and mixtures of polyethylene and polypropylene. The cover layer may also contain a pigment such as titanium dioxide. The material of the compensation layer advantageously is comprised of a nonwoven material. The nonwoven material may include polymerized alkene and/or bicomponent fibers. The compensation layer may also be coated with a surface-active substance which may contain silicone, for example, on the surface facing the storage layer. The primary storage layer may consist, for example, of a UCTAD material (UCTAD=uncreped through air-dried material), tissue wadding or a polymeric alkene. The primary storage layer is advantageously composed so that its edge areas are folded in and mutually overlap.

Both the liquid-impermeable layer as well as the liquid-permeable layer may be made of a polymerized alkene, such as polyethylene, polypropylene or a mixture thereof. At least one adhesive element and/or an adhesive layer may be applied to the liquid-impermeable layer for attaching the absorbent article according to this invention to an item of clothing. In addition, the absorbent article according to this invention may also have wings arranged at the sides.

This invention is explained in greater detail below on the basis of drawings with the help of the accompanying list of reference notation, showing:

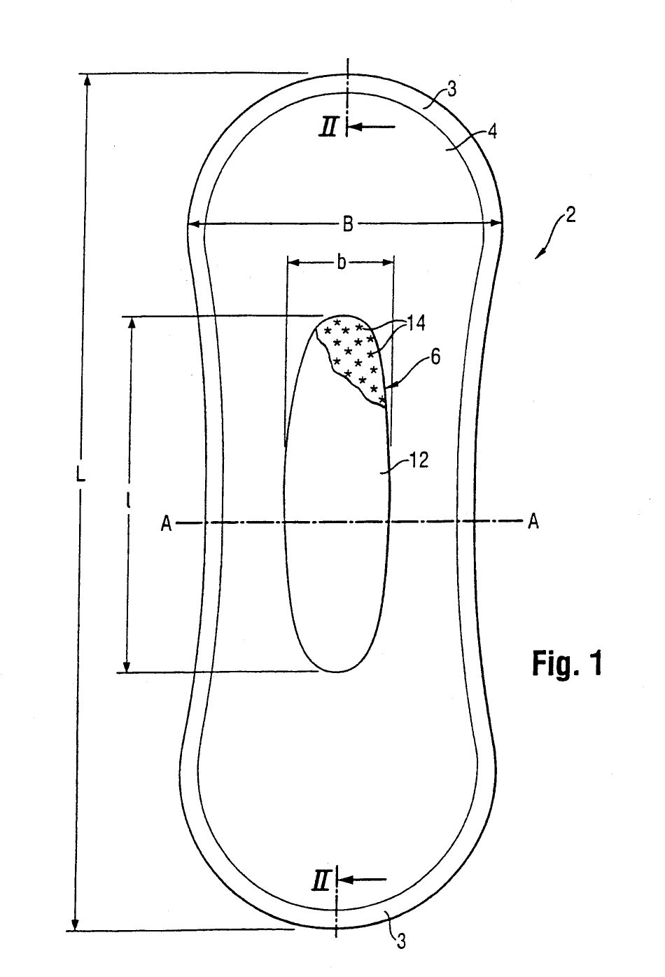

FIG. 1 a top view of a sanitary napkin;

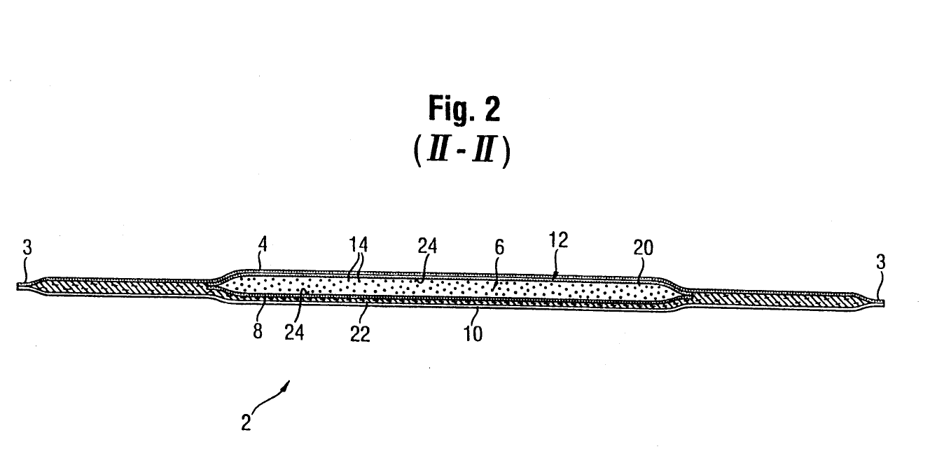

FIG. 2 a cross section along line A-A through the sanitary napkin according to claim 1;

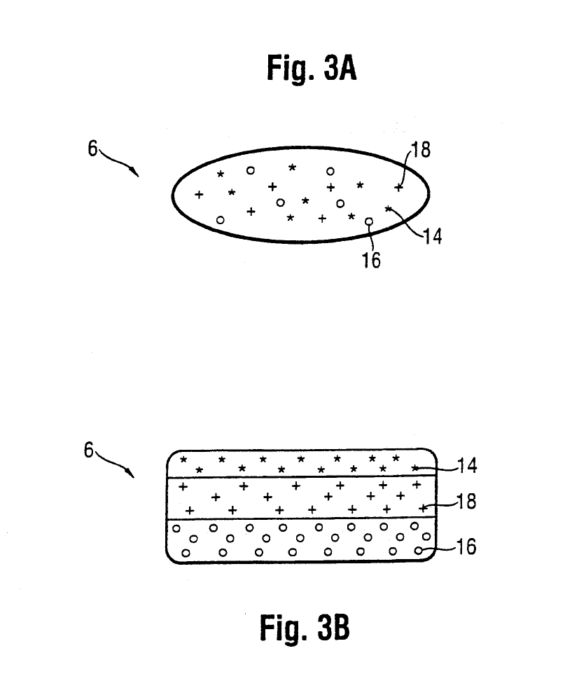

FIG. 3A a cross section through an absorbent body containing fiber material, superabsorbent material and cellulose fibers according to this invention which are at least partially in the form of granules;

FIG. 3B a cross section through an absorbent body containing fiber material, superabsorbent material and cellulose fibers according to this invention which are at least partially in the form of granules in a layered arrangement;

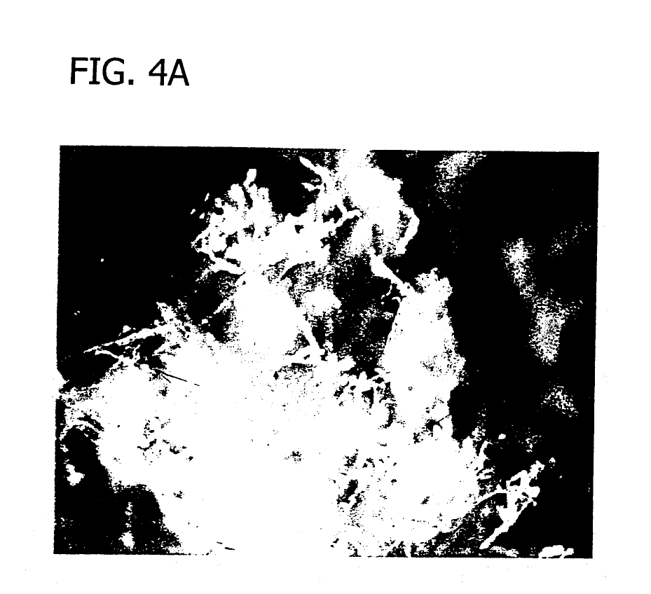

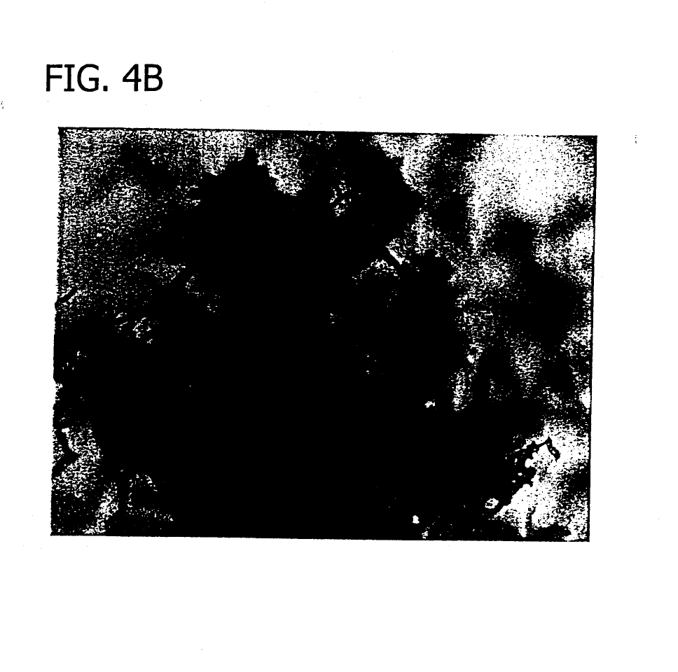

FIG. 4A a dark field micrograph of the cellulose fibers in fiber form (magnification 60×);

FIG. 4B a bright field micrograph of the fibers according to FIG. 4A (magnification 60×);

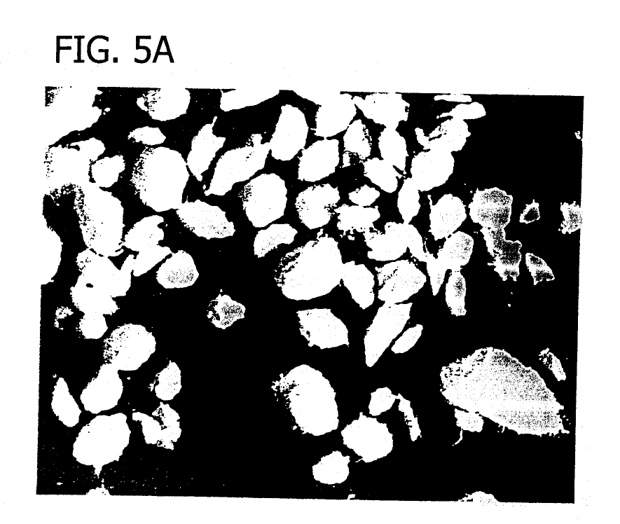

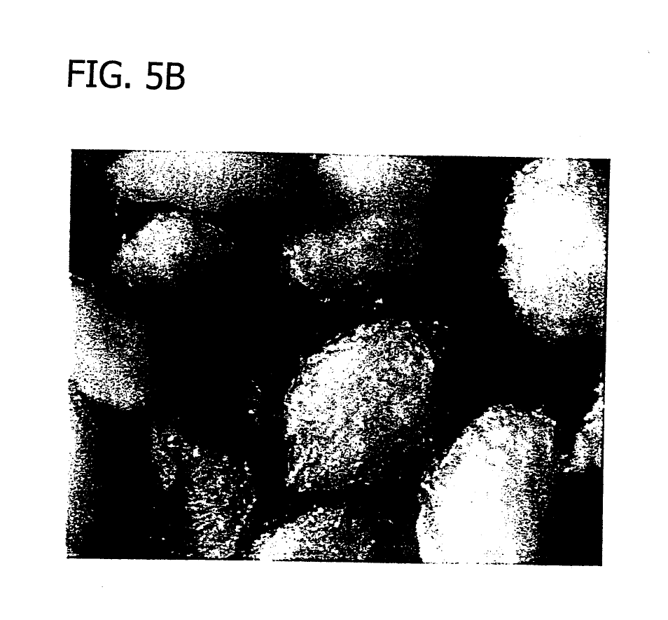

FIG. 5A a dark field micrograph of the cellulose fibers which are at least partially in the form of granules, shown here in the form of granules (magnification 14×);

FIG. 5B a dark field micrograph of the cellulose fibers which are at least partially in the form of granules according to FIG. 5A (magnification 60×);

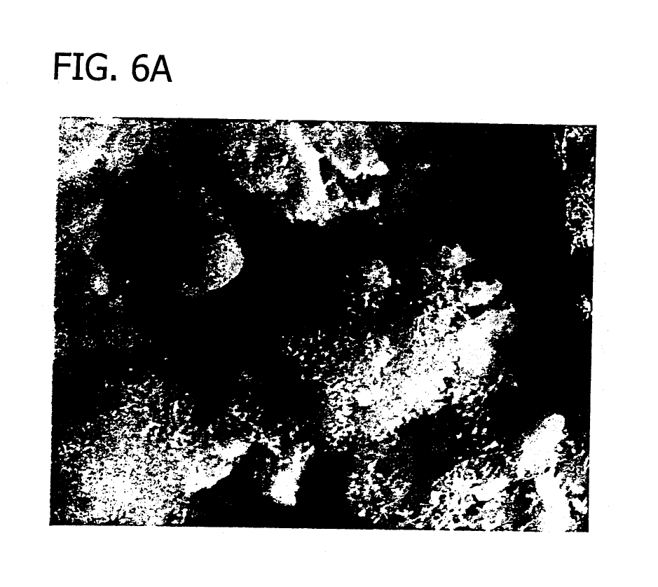

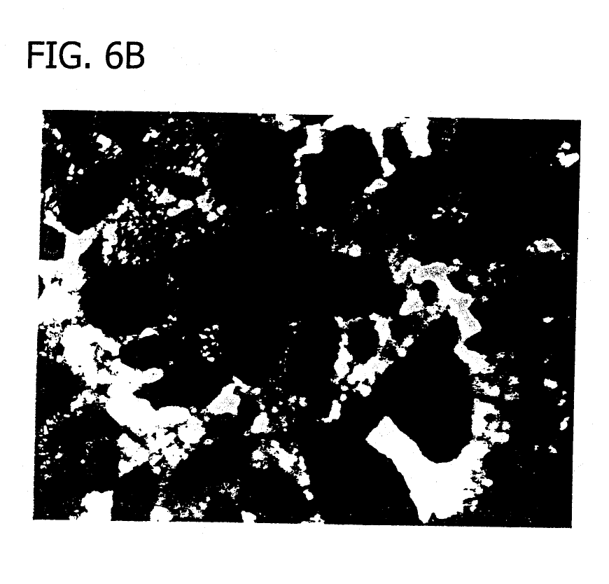

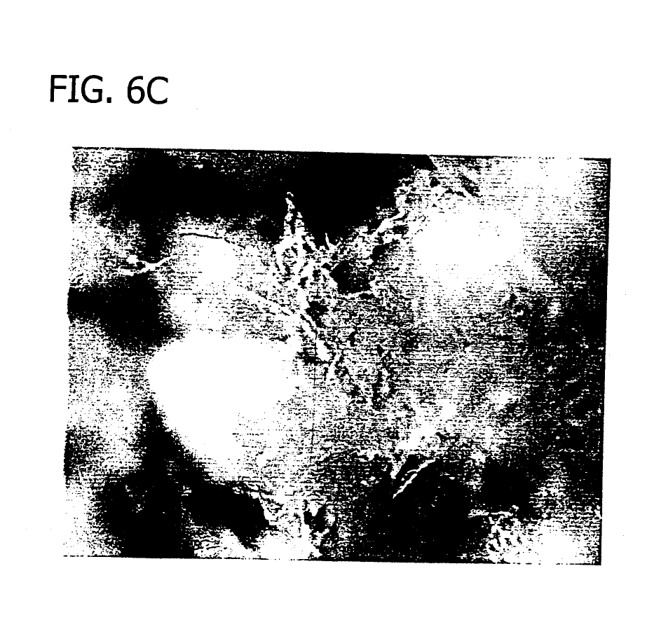

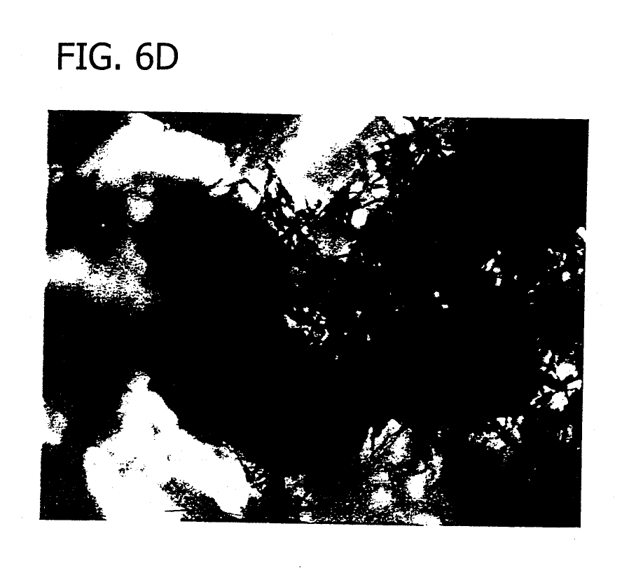

FIG. 6A a dark field micrograph of a mixture of fibrous and granular cellulose fibers which are present at least partially in the form of granules according to the present invention, mixing ratio of granules to fibers=2:1 (weight ratio) (magnification 14×);

FIG. 6B a bright field micrograph of a mixture as shown in FIG. 6A (magnification 14×);

FIG. 6C a dark field micrograph of a mixture as shown in FIG. 6A (magnification 50×);

FIG. 6D a bright field micrograph of a mixture as shown in FIG. 6A (magnification 50×);

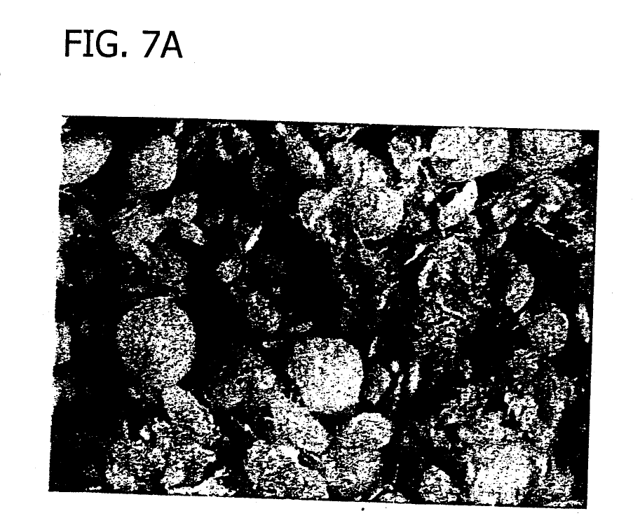

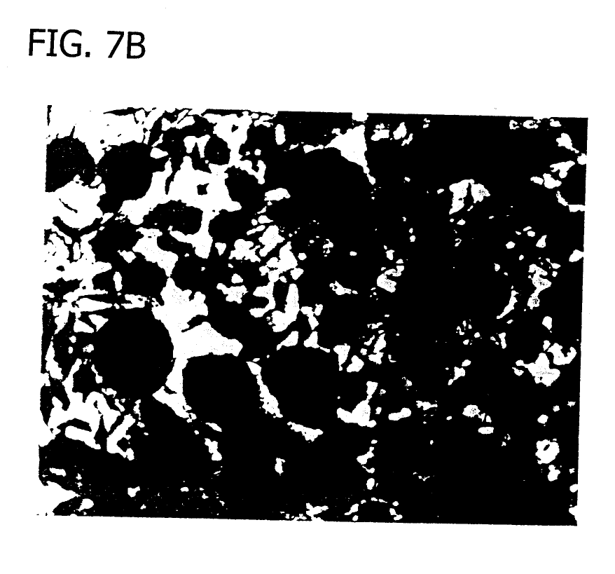

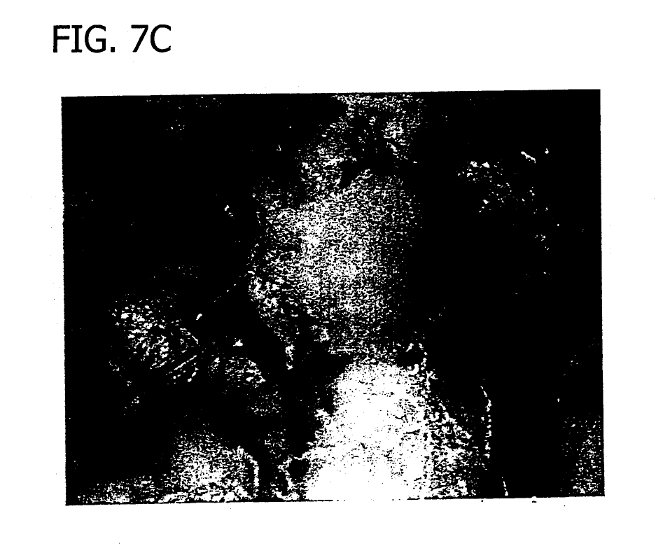

FIG. 7A a dark field micrograph of the cellulose fibers according to this invention which are at least partially in the form of granules, shown here as granules with fibers at the surface (magnification 14×);

FIG. 7B a bright field micrograph of the cellulose fibers according to this invention which are present at least partially in the form of granules, as shown in FIG. 7A (magnification 14×);

FIG. 7C a dark field micrograph of cellulose fibers present at least partially in the form of granules, as shown in FIG. 7A (magnification 60×);

FIG. 7D a bright field micrograph of the cellulose fibers which are at least partially in the form of granules as shown in FIG. 7A (magnification 60×);

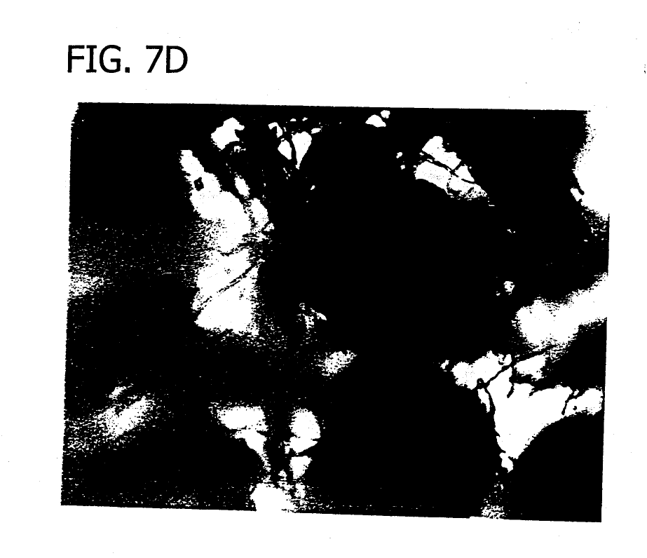

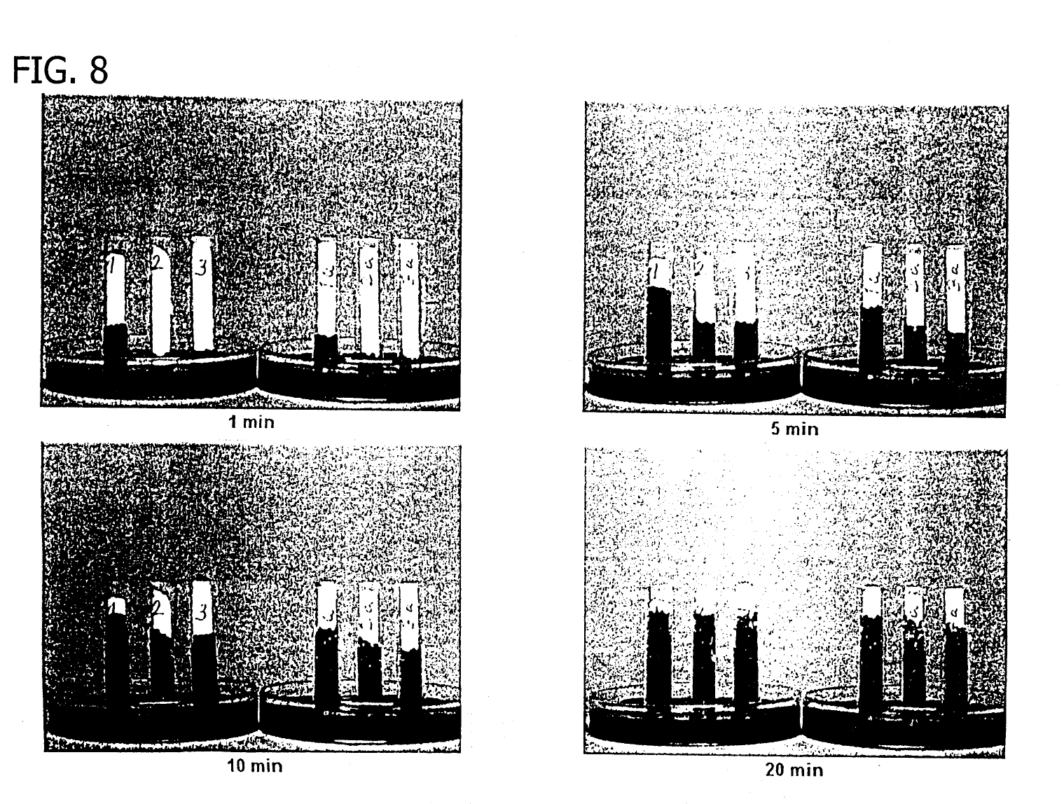

FIG. 8 a comparative diagram of the heights of rise with blood for various cellulose fibers according to this invention which are at least partially in the form of granules after one minute, five minutes, ten minutes, twenty minutes, where:
1) 1, 1a=granular form
2) 2, 2a=fiber form
3) 3, 3a=granular form with fibers at the surface, ratio of granules to fibers=2:1 (weight ratio);

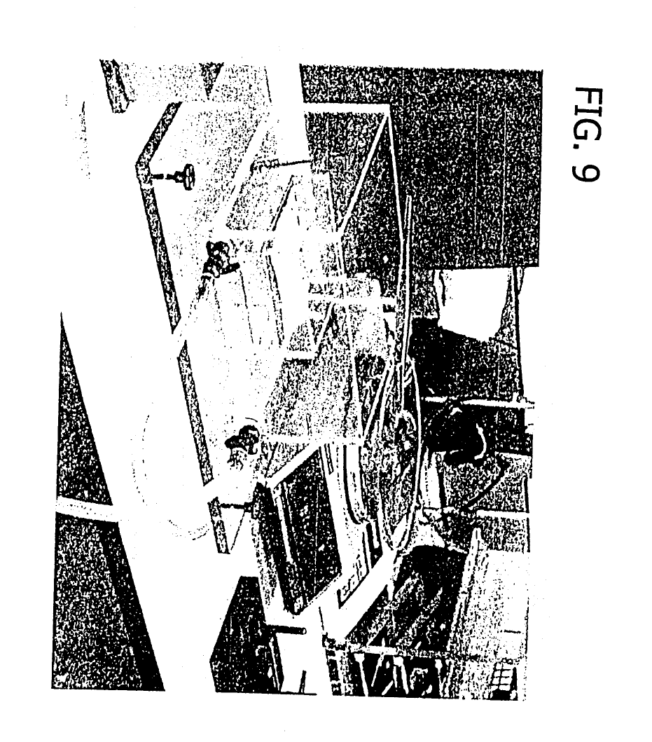

FIG. 9 a device for measuring liquid uptake; and

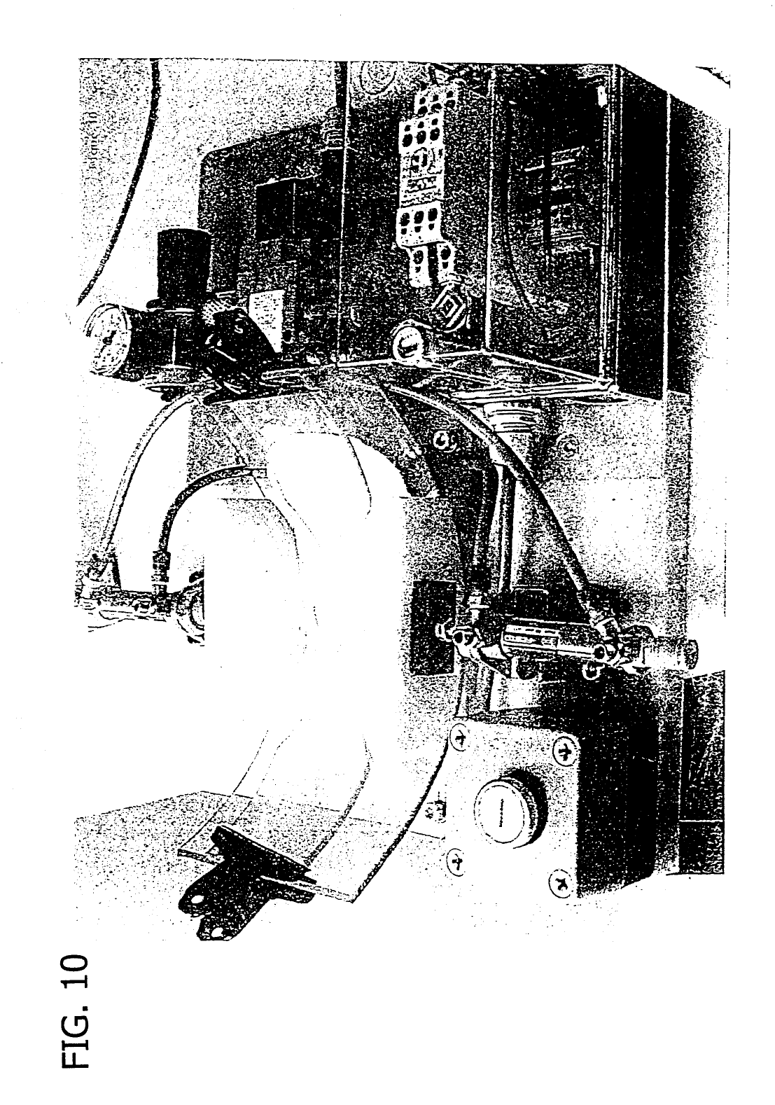

FIG. 10 a device for measuring dynamic rewetting, and

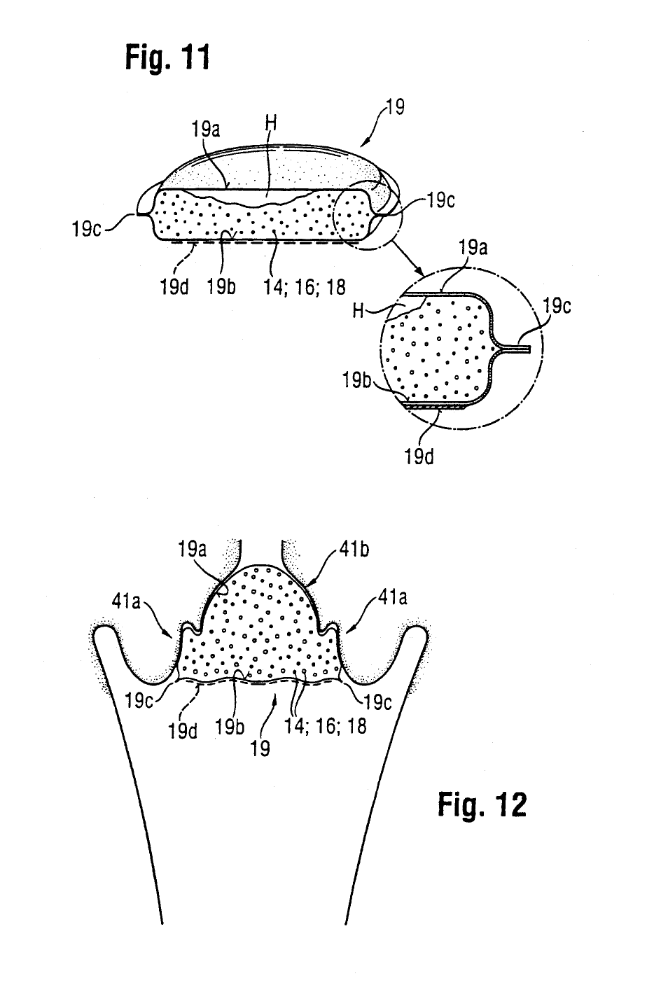

FIGS. 11 to 23 further embodiments of interlabial articles, usually in perspective view, wherein the front part is separated off, forming a vertical section view.

Although the absorbent articles according to this invention are shown in detail below by means of sanitary napkins, it is clear that the present invention is not limited to sanitary napkins, but instead comprises all absorbent hygiene products.

FIGS. 1 and 2 show a top view and a cross-sectional view of a sanitary napkin 2 according to this invention, having a front area, a middle area and an end area. The liquid-permeable layer 4 facing the wearer's body when the sanitary napkin 2 is worn and the liquid-impermeable layer 10 facing away from the wearer's body are bonded to one another in the edge area 3 of the sanitary napkin. A core piece 12 of the absorbent body 6 extends centrally in the longitudinal direction of the sanitary napkin 2, causing the liquid-permeable layer 4 to be elevated in the central area of the sanitary napkin in comparison with the front area and the end area. The core piece 12 of the absorbent body of the article according to this invention is arranged in the central area of the article and extends in the longitudinal direction of the article. The core piece 12 includes a (liquid-permeable) sheathing 24 made of a nonwoven material. The material which remains free-flowing even after coming in contact with a liquid is enclosed in the sheathing; in the present case, the free-flowing material is, for example, cellulose fibers according to this invention in granular form which are at least partially in the form of granules 14 having a particle size between 100 and 1000 μm, where the individual particles are largely egg shaped.

The length l of the core piece 12 is smaller than or equal to the length L of the absorbent article, and the width b of the core piece 12 is smaller than or equal to the width B of the absorbent article in this case.

In the embodiment illustrated in FIG. 2, the sheathing 24 is filled almost completely with material, which does not pose any problems because this material does not swell even when it comes into contact with a liquid, and thus there is no risk of the sheathing becoming too tight.

The liquid-permeable layer 4 and the liquid-impermeable layer 10 (panty shield) are bonded together in the edge area 3.

Core piece 12 of the absorbent body which is designed in the shape of an oval also has an absorbent secondary storage layer 8 containing cellulose beneath it in the embodiment of the absorbent article according to this invention illustrated in FIG. 2. This material containing cellulose first ensures wearing comfort and also serves as a reserve storage (secondary storage) for the case when the storage capacity of the core piece filled with material which remains free-flowing even after coming in contact with a liquid is exceeded. However, this reserve storage capacity need not usually be claimed because studies have shown that, by far, the majority of sanitary napkins are exposed to less than 5 ml fluid and the storage capacity of the core piece is sufficient for this amount in any case.

When absorbent article 2 according to this invention comes in contact with blood, the blood then penetrates through the liquid-permeable layer 4, penetrating through the sheathing into the core piece 12 with material 20, where it is retained.

In the edge area, the liquid-permeable layer 4 and the liquid-impermeable layer 10 are bonded together. In the present case, the bond is produced by gluing the layers using an adhesive. However, it is also possible to join the layers in some other way, e.g., by ultrasonic welding or heat sealing. Just as in the edge area of the article, two layers may also be glued together in the edge area of the sheathing.

An absorbent material 22 may also be present in the secondary storage layer; like absorbent material 20, it may contain cellulose fibers according to the present invention which are present at least partially in the form of granules.

Filling of the absorbent bodies with cellulose fibers according to the present invention which are present at least partially in the form of granules may be accomplished by the method described in PCT Application WO 98/43684.

FIG. 3A shows an absorbent body 6 which is filled with superabsorbent material 16 (o), fiber material 18 (+) and cellulose fibers 14 according to the present invention (*) which are present at least partially in the form of granules. According to this embodiment, they are present in the form of a true mixture.

An alternative embodiment is shown in FIG. 3B, according to which a layer 14 with cellulose fibers according to the present invention, which are present at least partially in the form of granules, is provided on the side of the absorbent body 6 facing the wearer's body when wearing the absorbent article 2, while a layer 16 of superabsorbent material is provided on the side of the absorbent body 6 facing away from the wearer's body when wearing the absorbent article 2. A fiber layer 18 is arranged between these two layers. Other layer sequences and arrangements are of course also possible.

FIGS. 4A and 4B show the cellulose fibers according to the present invention in the form of fibers in dark field and bright field micrographs, respectively. The fine cellulose fibers can be seen clearly. In particular when the retention capacity of an absorbent article is to be improved, the fibers illustrated in FIG. 4 can lead to an advantageous improvement in same when mixed with the granules according to this invention.

Accordingly, FIGS. 5A and 5B show, in different magnifications, the granules according to the present invention. FIG. 5B in particular shows very clearly the spherical or ovoid shape of the granular particles which look like small cotton swabs or balls. Negligibly small fiber residues projecting out of the granules can be seen on the surface. The granular form illustrated in FIG. 5 is especially suitable if the free-flowing property or the flow properties of an absorbent material in an absorbent article are to be improved.

FIGS. 6A through 6D show dark and bright field micrographs of a mixture of the cellulose fibers according to this invention which are at least partially in the form of granules, shown here in the form of granules and in the form of fibers in different magnifications. The ratio of the granular form to the fiber form is 2:1 (weight/weight).

The fiber form and the granular form differ clearly from one another according to these illustrations. This also shows that a good mixture of the two forms can be achieved. A mixing ratio of granular form to fiber form of 2:1 is especially preferred. Other suitable mixing ratios of granular form to fiber form include 60-70 wt % granules to 30-40 wt % fibers. The ratio of granules to fiber form depends of course on the intended effect which is to be achieved by the cellulose fibers according to the present invention which are present at least partially in the form of granules. If the free-flowing property in particular is to be improved, there should be a high percentage of granules, while a high percentage of fiber form would be preferable if rewetting is to be improved in particular.

Finally, FIGS. 7A through 7D show an especially preferred embodiment of the cellulose fibers according to the present invention which are present at least partially in the form of granules. Here again, the cellulose fibers according to the present invention which are present at least partially in the form of granules are shown in dark and bright field microscopy and in different magnifications, but in this case, fibers are present on the surface of the granular particles. These fibers are much longer than the short fiber residues shown on the granular particles in FIG. 5.

The cellulose fibers according to the present invention which are present at least partially in the form of granules are preferably prepared, as illustrated in FIGS. 7A through 7D according to a method wherein the following steps are carried out:
a. producing fine cellulose fibers,
b. winding the fibers up to form granules or coagulating the fibers in a moist atmosphere to form granules, where the process of winding and/or coagulating is interrupted so promptly as to yield granules with incompletely wound fibers on their surface.

FIG. 8 shows the various heights of rise of blood (sheep's blood is used here because it corresponds to human blood in all properties that are essential here) in the cellulose fibers according to the present invention which are present at least partially in the form of granules. A good height of rise is found for all the embodiments presented here. It can also be seen from this experiment that the granular material has an especially good height of rise which is apparent after about five minutes in comparison with the fiber material alone or the mixture.

The following FIGS. 11 to 23 show further embodiments of the invention in the form of interlabially usable female hygiene articles, in the following called ‘interlabial product’ for short, respectively.

FIG. 11 shows a magnified view of an interlabial product 19, in perspective view and cut, consisting of a bodyside, proximal liquid-permeable cover layer 19a and a liquid-impermeable distal backing layer 19b/19d facing away from the body, which are joined together in the edge areas 19 c. In this case, 19d is a liquid-impermeable coating which does not reach the area of the mucous membranes of the wearer. Cavity H is, at least partially, filled with the granules or cellulose fibers 14, 16, 18 etc. of the invention.

FIG. 12 shows a magnified and schematic sectional view of the interlabial product 19 in use, i.e., as an arrangement embedded in the labiae portion 41a and the vestibule 41b, ensuring high wear comfort. In this case, the liquid-impermeable layer 19d does not reach the area of the mucous membranes of the wearer.

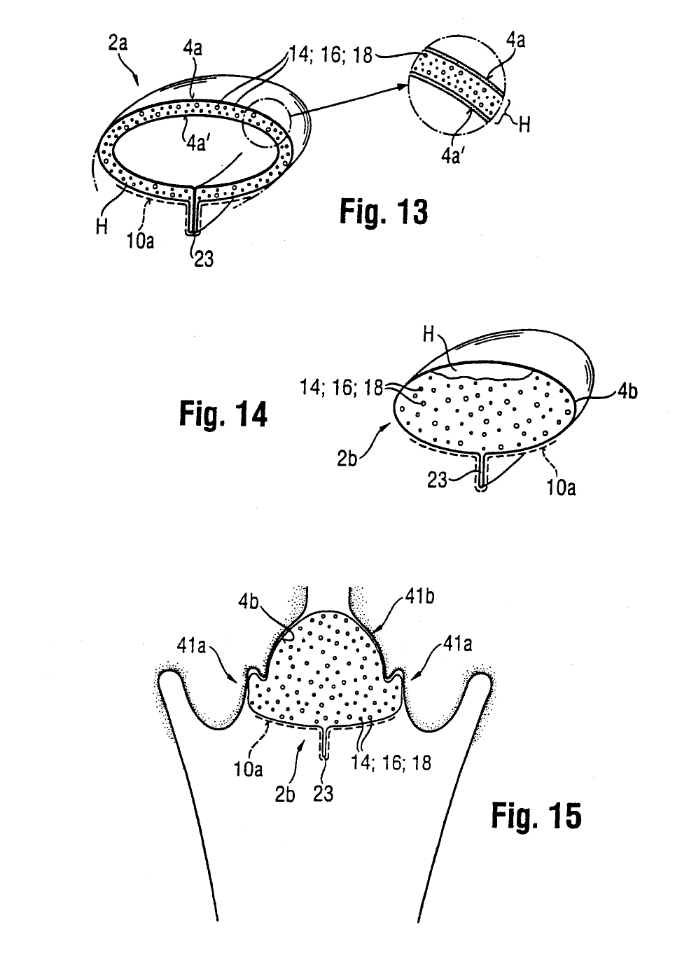

FIG. 13 shows a perspective, cut view in magnified detailed section of a further interlabial product 2a consisting of a two-plied layer 4a, 4a′, wherein the two layers 4a, 4a′ encompass a cavity H formed therein, which is, at least partially, filled with the granules or cellulose fibers 14, 16, 18, wherein the two-plied layer 4a, 4a′ shows a mushroom-shaped cross-section, and the (distal) ends facing away from the body are formed into a joint 23, for example by U-welding, wherein a handle is formed. In the (distal) portion facing away from the body, a liquid-impermeable coating and/or lacquer coat 10a is applied, but only to the extent that the mucous membranes of the labiae portions 41a etc. cannot be impinged thereby.

FIG. 14 shows a further interlabial product 2b having a one-walled exterior layer 4a and, similar to what was described above, formed into a mushroom-shaped body, whereby a cavity H is formed. The cavity H is at least partially filled with granules or cellulose fibers 14, 16, 18 etc. The ends of the layer 4a are distally welded together or glued into a joint 23 in a manner facing away from the body, whereby a handle is formed. A layer 10a causing the impermeability with respect to liquid is applied in the lower region without reaching the mucous membranes of the wearer.

In FIG. 15, the use of the interlabial product 2b according to FIG. 14 is shown. The embedding into the labiae zones 41a and the vestibule is a full-areal embedding, and therefore provides highest wear comfort. The liquid-impermeable coating 10a only remained in the (distal) area facing away from the body in this case. The handling of the removal of the saturated absorbent body is ensured by the handle at 23.

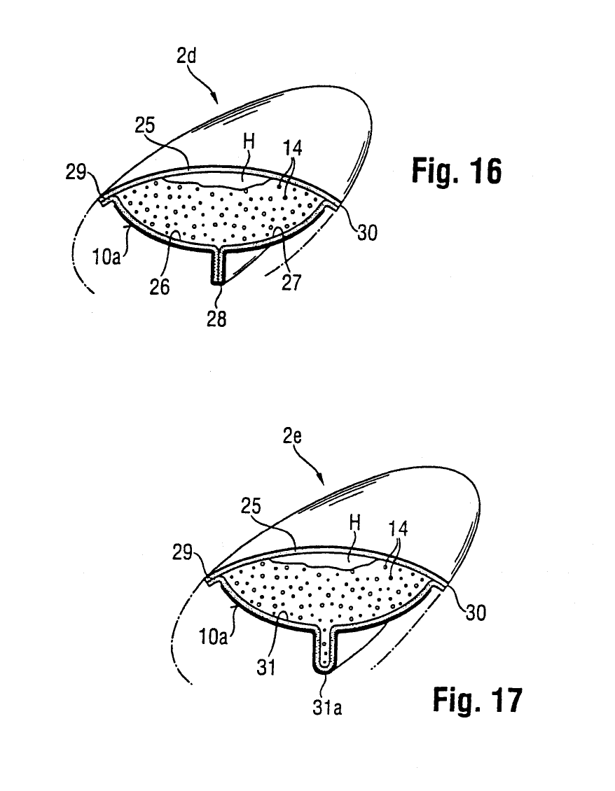

The embodiment of FIG. 16 shows an interlabial product 2d, the sheathing of which is formed by a top part 25 and two bottom parts 26 and 27. The joints or seams are designated with 28, 29 and 30. The filling with granules is referred to as 14, and the liquid-impermeable coating as 10a.

The embodiment of FIG. 17 corresponds to the product of FIG. 16, however, the sheathing of this product 2e is only formed of two parts 25 and 31. The bottom part 31 is offset or folded here, forming a handle 31a.

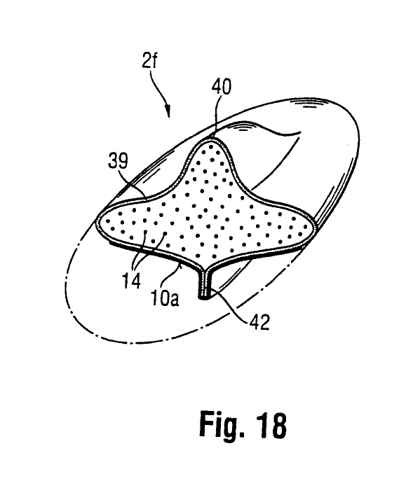

The embodiment of FIG. 18 shows an interlabial product 2f corresponding to the construction of FIG. 17, but with the difference that not only the bottom part of the sheathing, but also the top cover layer 39 shows an offset portion, forming a dome in the form of a hill-shaped raised portion 40, which is provided for accommodation in the vestibule 41b according to FIG. 12.

Forming a handle 42, a connection of the two ends in the form of a welded joint is provided, and a liquid-impermeable layer 10a is laminated on top.

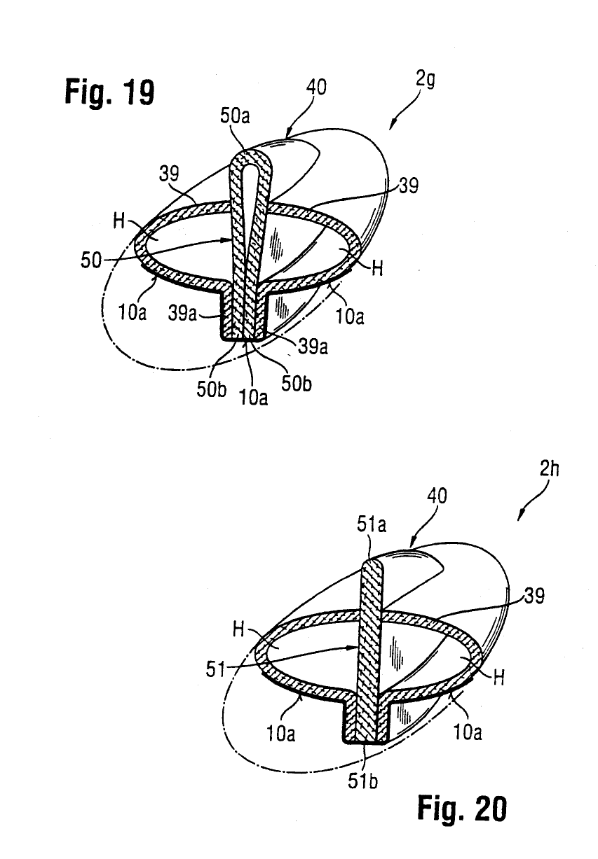

FIG. 19 shows an interlabial product 2g without filling, consisting of two parts 39 with branches 39a and 39b, which are put together vertically to each other, forming a hill or vestibule part 40 by means of the formation of a loop 50a and a handle part 50b, wherein the loop-shaped vertical part (50, 50 a, 50 b) is formed of one part into two plies by means of folding.

A respective construction 2h of an absorbent body 2h is shown in FIG. 20. In this case, the absorbent vertical part 51 is formed as one ply in the form of an absorbent pad and forms on top a raised portion 40 which reaches into the vestibule, and a handle 51b on the bottom.

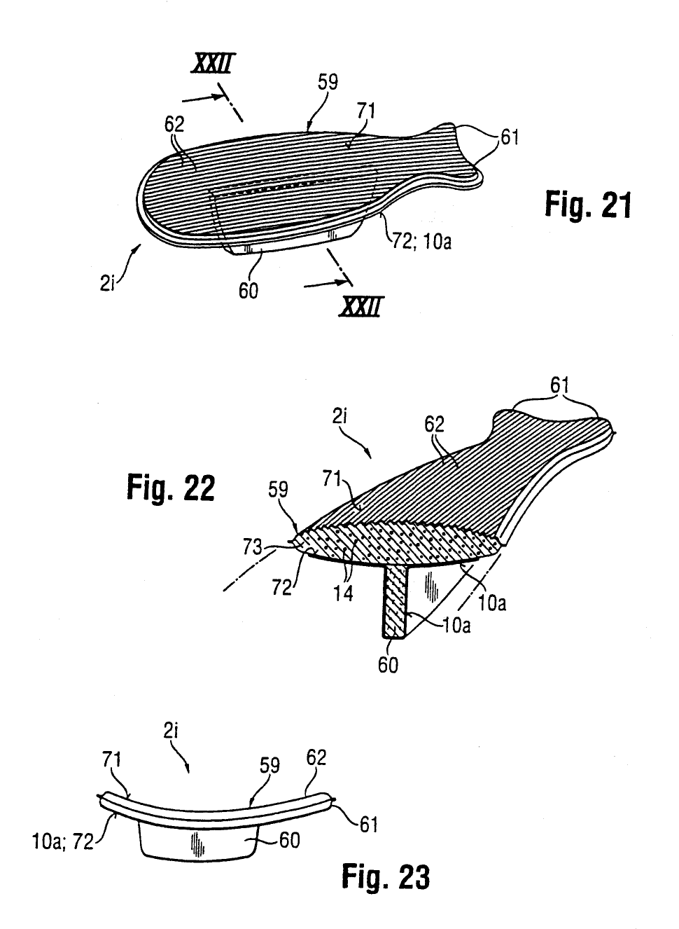

FIGS. 21 to 23 show an interlabial product 2i in the form of a fish, wherein the fish-body-trunk-shaped portion 59 forms the main absorbent body, and the fin-shaped portion 60 forms the additional absorbent body and the handle. The trunk-shaped portion is formed as a flat pad 59, wherein, between the cover layer 71 and the backing layer 72, a layer 73 of cellulose fibers at least partially in the form of granules is arranged. An extremity 61 can have a tail-fin shape. Both parts 59 and 60 are, for example, blunt and glued together in a T-shape or welded by ultrasonic welding. The liquid-impermeable coating 10a applied on the bottom contributes to enhance the stability of the connection. To enlarge its functional area, the absorbent area of the trunk-shaped portion 59 can have a pleating 62, which also serves to improve the softness (comfort).

As shown in the side view in FIG. 23, the interlabial product 2i can be adapted to the anatomy of the female pelvic floor or be correspondingly pre-formed in a concave manner.

### EXAMPLES

To demonstrate some of the advantageous effects of the present invention, a number of experiments were conducted, as explained below.

Test Methods Used

Absorbency/uptake Capacity for Sanitary Napkins, Test Method

This test method determines the total absorbency of a test object obtained from a hygiene article. Two values are determined, the absorbency with and without a weight load.

Equipment and Materials:

i. Liquid uptake apparatus (see FIG. 9)
ii. Plexiglass frame screwed on a screen, 100×100 mm, height 20 mm
iii. Plexiglass ram, 99×99×25 mm
iv. Stopwatch
v. Large microscope slide
vi. Weight, 250 g, 100×100 mm base area
vii. Pincers
viii. Aluminum plate 100×65 mm, to hold down sample and prevent it from floating
ix. Scales
x. Test liquid or sheep's blood

The absorbency of a sanitary napkin for menstrual use is measured as follows:

The middle section of a sanitary napkin (length 100 mm) is weighed and placed in a plexiglass container with a metal screen bottom. Then place this in a container with the dimensions 200×200×100 mm. Next add approximately two liters of a model menstrual fluid (or sheep's blood) so that the sample is covered completely with liquid. Cover with the aluminum plate. Drain off the liquid after a waiting time of ten minutes. Remove the sample with the pincers and weigh on a microscope slide. After reweighing, this yields the absorption capacity without loading by taking the difference.

Then return the sample to the container and load with a 2.5 kg weight with a base area of 100×100 mm, add liquid again and wait ten minutes. After the waiting time, drain off the liquid and weigh the sample again. This value indicates the absorption capacity with loading.

This value is expressed as specific absorption per gram and in blood or blood substitute uptake per section of sanitary napkin.

Dynamic Rewetting, Test Method

This test method determines the amount of liquid released from the surface of the sanitary napkin to a medium (filter paper) after a sanitary napkin is exposed to a given quantity of test fluid. Pressure is applied dynamically from the sides to simulate the pressure applied by the thighs while the sanitary napkin is being worn.

Equipment and Materials:

xi. Rewetting apparatus with mechanical pressure at the side (FIG. 10)
xii. Elongated hole stencil
xiii. Two clamps
xiv. Eppendorf pipette, 1 ml
xv. Sheep's blood
xvi. Stopwatch and alarm
xvii. Half-round pan for rewetting
xviii. 2.5 kg weight

Stack of Filter Paper, Schleicher & Schüll 2040A

Scales

Measurement System for Determination of Area

Procedure:
xix. Determine the dry weight of the filter paper stack.
xx. Remove the silicone paper from the sanitary napkin and cover with a layer of tissue.
xxi. Place the sanitary napkin in the apparatus and place the elongated hole stencil on the napkin. Using the clamps, attach both ends of the stencil to the substrate.

Using the Eppendorf pipette, place 1 ml blood in the elongated hole and start timing with the stopwatch at the same time.

After 15 seconds, start the compression by pushing on a button (the jaws open again automatically). Immediately after opening, add 1 ml blood again, wait 15 seconds and compress. Repeat this procedure until a total of 5 ml blood has been added and the sample has been compressed five times. Then set the alarm for ten minutes.

After waiting ten minutes, remove the clamps and stencil and place the napkin in the half-round pan, preferably without deforming it.

Then place the stack of filter paper on the napkin and put the 2.5 kg weight on top for 15 seconds (half-round weight+500 g weight).

After 15 seconds, determine the wet weight of the filter paper stack and the rewetting area (cm²). The difference between the wet weight and the dry weights yields the rewetting amount (g).

Report of Findings:

Rewetting is given in grams. In addition, the area can be measured with another device (camera). The absorption (penetration) times for the respective 1 ml doses are also given.

Results

The following table (Table 1) summarizes the results of the above tests.

In this table, LC200 denotes cleaned and mechanically disintegrated cellulose fibers from alpha-cellulose, from J. Rettenmaier & Söhne GmbH & Co., Germany, and LC 200 HF denotes granules prepared from these fibers (particle size 400 μm, powder density 210 g/l±15%), also from the same company. PMH is polymethylene urea and SAP stands for superabsorbent particles.

In summary, it can thus be stated that the cellulose fibers according to the present invention which are present at least partially in the form of granules have the advantages mentioned in the detailed description. The accompanying figures and examples should serve only to illustrate this invention without in any way restricting the scope of the embodiments presented here.

TABLE 1

| | | | | 2:1 mixture | | | |
|---|---|---|---|---|---|---|---|
| | | LC200 HF | LC200 | Granulated | | | |
| | | Granulated | Cellulose | cellulose fibers/ | | | Cellulose, |
| | | cellulose fibers | fibers | cellulose fibers | PMH | SAP | fiberized |
| Height of rise | mm | | | | | | |
| (dimensions of acrylic | | | | | | | |
| tube, 50 mm high, | | | | | | | |
| inside diameter 6 mm, | | | | | | | |
| bottom of Perlon mesh) | | | | | | | |
| After 1 minute | mm | 24 | 12 | 12 | 22 | 0 | 10 |
| After 5 minutes | mm | 37 | 27 | 27 | 40 | 0 | 15 |
| After 10 minutes | mm | 43 | 36 | 34 | 47 | 2 | 28 |
| After 20 minutes | mm | 44 | 43 | 43 | 50 | 2 | 40 |
| Absorbency | g/g | | | | | | |
| Without load | g/g | 13-16 | 13-16 | 13-16 | 12-18 | 18-20 | 13-16 |
| With load | g/g | 6-8 | 6-8 | 6-8 | 8-12 | 13-16 | 6-8 |
| Dynamic rewetting | g | | | | | | |
| Rewetting amount | g | 0.01-0.4 | 0.2-0.6 | 0.01-0.4 | 0.02-0.4 | 0.4-0.9 | 0.2-0.8 |

List of Reference Numbers
L length of the absorbent article 2
l length of the core piece 12
B width of the absorbent article 2
b width of the core piece 12
H cavity
2 absorbent article
3 edge area (FIG. 1)
2a to 2h interlabial absorbent article (FIGS. 11 to 23)
4 liquid-permeable cover layer
4a liquid-permeable first cover layer (FIG. 13)
4a′ liquid-permeable second cover layer (FIG. 13)
6 absorbent body
8 secondary storage layer
10 liquid-impermeable backing layer
10a liquid-impermeable coating
12 core piece
14 granules according to this invention (cellulose fibers)
16 superabsorbent material
18 fiber material
19 interlabial product (FIG. 11)
19a cover layer, permeable
19b backing layer
19c edge area
19d coating, impermeable
20 absorbent material
22 absorbent material
23 connection, weld, seam
24 liquid-permeable sheathing of the core piece 12 (FIG. 2)
25 liquid-permeable cover layer
26 bottom-layer part (FIG. 16)
27 bottom-layer part (FIG. 16)
28 weld, connection
29 weld, connection
30 weld, connection
31 backing layer (FIG. 17)
31a handle (FIG. 17)
39 cover layer (FIG. 18)
39a branched-off backing-layer part
40 absorbent-body hill
41 vestibule (FIG. 17)
41a labiae zone
41b vestibule
42 connection, weld (FIG. 18)
50 vertical loop insertion part, folded (FIG. 19) for forming a vestibule absorbent body 50a and a handle 50b
50a vestibule absorbent body
50b handle part (ends of 50 put on top of each other)
51 vertical insertion part, one piece, unfolded (FIG. 20)
51a vestibule absorbent body
51b handle part
59 fish-body shaped portion (FIGS. 21 to 23), pad
60 ventral fin, for forming a fish shape and a handle
61 extremity, for forming a fish-tail shape
62 pleating
71 cover layer of 59
72 backing layer of 59
73 layer of cellulose fibers 14

What is claimed is:

1. An absorbent article (2) comprising cellulose fibers (14) present at least partially in the form of granules.

2. The absorbent article (2) according to claim 1, wherein the cellulose fibers (14) present at least partially in the form of granules are obtained from wood and/or other plant fibers.

3. The absorbent article (2) according to claim 1 or 2, wherein the absorbent article comprises at least the following additional components:
a liquid-permeable cover layer (4) facing the wearer's body when the article is being worn,
a liquid-impermeable backing layer (10) facing away from the wearer's body when the article is being worn, and
an absorbent body (6) arranged between the liquid-permeable cover layer (4) and the liquid-impermeable backing layer (10), where the cellulose fibers (14) present at least partially in the form of granules are arranged in the absorbent body (6).

4. The absorbent article (2) according to one or more of the preceding claims, wherein the cellulose fibers (14) present at least partially in the form of granules are completely present in granular form.

5. The absorbent article (2) according to claim 4, wherein the granules are free-flowing.

6. The absorbent article (2) according to claim 4 or 5, wherein the particles in the granules have an average diameter of 50 to 3000 μm.

7. The absorbent article (2) according to claim 6, wherein the beads in the granules have an average diameter of 400 μm.

8. The absorbent article (2) according to one or more of the preceding claims 4 through 7, wherein the granules have an average powder density of 30 to 600 g/l according to DIN 53,468.

9. The absorbent article (2) according to claim 8, wherein the granules have an average powder density according to DIN 53,468 amounting to 100 to 300 g/l±15%.

10. The absorbent article (2) according to one or more of claims 1 through 3, wherein the cellulose fibers (14) present at least partially in the form of granules are partially also present in the form of fibers.

11. The absorbent article (2) according to claim 10, wherein the fibers have an average fiber length of 100 to 600 μm.

12. The absorbent article (2) according to claim 11, wherein the fibers have an average fiber length of 300 μm.

13. The absorbent article (2) according to one or more of claims 10 through 12, wherein the fibers have an average fiber thickness of 10 to 50 μm.

14. The absorbent article (2) according to claim 13, wherein the fibers have an average fiber thickness of 20 μm.

15. The absorbent article (2) according to one or more of the preceding claims, wherein the fibers used to produce the granules are fine fibers, preferably fibers with an average fiber length of 100 to 600 μm and an average fiber thickness of 10 to 50 μm.

16. The absorbent article (2) according to one or more of the preceding claims, wherein the cellulose fibers (14) present at least partially in the form of granules are obtained from α-cellulose.

17. The absorbent article (2) according to one or more of the preceding claims, wherein the cellulose fibers (14) present at least partially in the form of granules are present in the form of a mixture of granules and fibers.

18. The absorbent article (2) according to claim 17, wherein the mixture is free-flowing.

19. The absorbent article (2) according to claim 17 or 18, wherein the mixture contains 20 to 90 wt % granules and 10 to 80 wt % fibers.

20. The absorbent article (2) according to claim 19, wherein the mixture contains 60 to 70 wt % granules and 30 to 40 wt % fibers.

21. The absorbent article (2) according to one or more of claims 17 through 20, wherein the fibers are present at the surface of the granules.

22. The absorbent article (2) according to one or more of the preceding claims, wherein an absorbent material (22) which serves as a secondary storage (8) is arranged on the side of the liquid-impermeable layer (10) facing the wearer's body.

23. The absorbent article (2) according to claim 22, wherein the material (22) which serves as the secondary storage is a coform material, an airlaid material, tissue wadding and/or a nonwoven material, in particular a spunbonded nonwoven or a carded nonwoven.

24. The absorbent article (2) according to one or more of the preceding claims, wherein at least one third of the absorbent material (20, 22) of the absorbent body (6) consists of the cellulose fibers (14) present at least partially in the form of granules.

25. The absorbent article (2) according to claim 24, wherein at least half of the absorbent material (20, 22) consists of the cellulose fibers (14) present at least partially in the form of granules.

26. The absorbent article (2) according to claim 25, wherein at least two-thirds of the absorbent material (20, 22) consists of the cellulose fibers (14) present at least partially in the form of granules.

27. The absorbent article (2) according to claim 26, wherein at least 80% of the absorbent material (20, 22) consists of the cellulose fibers (14) present at least partially in the form of granules.

28. The absorbent article (2) according to claim 27, wherein the absorbent material (20, 22) consists of the cellulose fibers (14) present at least partially in the form of granules.

29. The absorbent article (2) according to one or more of the preceding claims 1 through 27, wherein the absorbent material (20, 22) comprising the cellulose fibers (14) present at least partially in the form of granules is incorporated into a matrix of fiber material.

30. The absorbent article (2) according to claim 29, wherein the absorbent material (20, 22) comprising the cellulose fibers (14) present at least partially in the form of granules is blended homogeneously into the fiber material.

31. The absorbent article (2) according to claim 29, wherein the absorbent material (20, 22) comprising the cellulose fibers (14) present at least partially in the form of granules is incorporated between layers of fiber material (18).

32. The absorbent article (2) according to one or more of claims 29 through 31, wherein the fiber material (18) is cellulose, a cellulose/polypropylene mixture and/or a coform material.

33. The absorbent article (2) according to one or more of claims 29 through 32, wherein the ratio of absorbent material (20, 22) to fiber material (18) is from 1 to 25 wt % to 99 to 75 wt %.

34. The absorbent article (2) according to claim 33, wherein the ratio of absorbent material (20, 22) to fiber material (18) is from 5 to 25 wt % to 95 to 80 wt %.

35. The absorbent article (2) according to claim 34, wherein the ratio of absorbent material (20, 22) to fiber material (18) is from 10 to 15 wt % to 90 to 85 wt %.

36. The absorbent article (2) according to one or more of the preceding claims 1 through 27 or 29 through 35, wherein the absorbent material (20, 22) comprises a superabsorbent material (16).

37. The absorbent article (2) according to claim 36, wherein the superabsorbent material (16) is a polyacrylate.

38. The absorbent article (2) according to one of claims 36 or 37, wherein the cellulose fibers (14) present at least partially in the form of granules and/or the fiber material (18) and/or the superabsorbent material (16) are arranged in layers.

39. The absorbent article (2) according to claim 38, wherein the fiber material (18) forms a layer between the superabsorbent material (16) and the cellulose fibers (14) present at least partially in the form of granules.

40. The absorbent article (2) according to claim 39, wherein the cellulose fibers (14) present at least partially in the form of granules are arranged on the side of the article facing the wearer's body, and the superabsorbent material (20, 22) is arranged on the side of the article facing away from the wearer's body.

41. The absorbent article (2) according to one of claims 36 or 37, wherein the cellulose fibers (14) present at least partially in the form of granules, the fiber material (18) and the superabsorbent material (16) are present in the form of a mixture.

42. The absorbent article (2) according to one or more of the preceding claims, wherein the cellulose fibers (14) present at least partially in the form of granules are coated with an agent such as dextrin which retards the uptake of liquid.

43. The absorbent article (2) according to one or more of the preceding claims, wherein the cellulose fibers (14) present at least partially in the form of granules have been rendered hydrophobic by suitable agents such as Wacker-Silicone Finish WS 60 E.

44. The absorbent article (2) according to one or more of the preceding claims, wherein the cellulose fibers (14) present at least partially in the form of granules have a water retention capacity of at least 6 g H₂O/g.

45. The absorbent article (2) according to one or more of the preceding claims, wherein the cellulose fibers (14) present at least partially in the form of granules have a water retention capacity of 8-16 g H₂O/g.

46. The absorbent article (2) according to one or more of the preceding claims, wherein the absorbent material (20, 22) contains at least one care substance.

47. The absorbent article (2) according to one or more of the preceding claims, wherein the absorbent body (6) has at least one core piece (12), in which the absorbent material (20, 22) is accommodated, where preferably the length l of the core piece (12) is less than or equal to the length L of the absorbent article, and the width b of the core piece (12) is less than or equal to the width B of the absorbent article.

48. The absorbent article (2) according to one or more of the preceding claims, wherein it is a feminine hygiene article.

49. The absorbent article (2) according to claim 48, wherein the feminine hygiene article is a sanitary napkin, in particular an ultrathin sanitary napkin and/or a panty liner.

50. The absorbent article (2) according to one or more of claims 1 through 47, wherein it is a diaper and/or an incontinence pad.

51. The absorbent article (2) according to one or more of the preceding claims 3 through 50, wherein the liquid-permeable layer (4) has a centrally located opening.

52. Use of cellulose fibers (14) present at least partially in the form of granules as defined in one or more of the preceding claims 1, 2 and 4 through 21 as a material in an absorbent article.

53. Use according to claim 52, wherein the cellulose fibers (14) present at least partially in the form of granules are present essentially in the form of granules, and where the flow properties of the absorbent material (20, 22) are improved.

54. Use according to claim 52, wherein the cellulose fibers (14) present at least partially in the form of granules are present essentially in the form of fibers, and the rewetting properties of the absorbent article are improved.

55. The absorbent article (2) according to one of claims 1 to 21, characterized in that the article is an interlabial absorbent body (19, 2a, 2b, 2d, 2e, 2f, 2g, 2h, 2i) (FIGS. 11 to 23).

56. The absorbent article according to claim 55, including a liquid-permeable cover layer (19a), a liquid-impermeable backing layer (19b, 19d) opposite thereto, which are connected to each other in the edge areas (19c), wherein a cavity (H) formed between the cover layer (19a) and the backing layer (19b, 19d) is at least partially filled with cellulose fibers (14) which are at least partially present in the form of granules (FIG. 11).

57. The absorbent article according to claim 56, characterized in that the backing layer is formed of a carrier layer (19b), which carries a liquid-impermeable coating (19d) (FIG. 11).

58. The absorbent article according to claim 57, characterized in that the coating (19d) is only disposed in the area of the backing layer of the interlabial absorbent article (19) which is directed to the outside during wear and is not to be brought into contact with the mucous membrane (FIG. 12).

59. The absorbent article according to one or more of claims 55 to 58, characterized in that the interlabial absorbent body (2a) is formed of a two-plied layer (4a, 4a′), wherein the two layers (4a, 4a′) encompass a cavity (H), which is preferably loosely filled with cellulose fibers (14) which are at least partially present in the form of granules, wherein the two-plied layer forms a mushroom shape, and the distal ends of the layers (4a, 4a′) are connected to each other at (23) in a way that they form a handle for introducing the interlabial absorbent body (2d) (FIG. 13).

60. The absorbent article (2b) according to one or more of claims 55 to 58, characterized in that a one-walled outer layer (4a) forms a mushroom-shaped body, which encloses a cavity (H), which is at least partially filled with the cellulose fibers (14) which are at least partially present in the form of granules (FIGS. 14 and 15).

61. The absorbent article (2d) according to one or more of claims 55 to 58, characterized in that the backside of the absorbent body sheathing is formed of two parts (26) and (27), which are connected to each other forming a handle at (28) (FIG. 16).

62. The absorbent article (2e) according to one of claims 55 to 58, characterized in that the backing layer (31) forms on its side disposed away from the body in the central portion an offset or fold (31a) facing away from the body, and which is formed as a handle (FIG. 17).

63. The absorbent article (2f) according to one of claims 55 to 62, characterized in that the interlabial absorbent article has a hill-shaped raised portion (40) disposed centrally on its surface and extending in longitudinal direction, which can be introduced into the vestibule (FIG. 18).

64. The absorbent article (2g) according to claim 63, characterized in that the raised portion is formed by a loop portion (50) of liquid-permeable material, which centrally penetrates the cover layer (39), and the rounded portion (50a) of which forms the raised portion (40), while the ends (50b) thereof put on top of each other extend between the material portion of the backing sheet parts (39a), the ends thereof being bent downwardly, and forming the handle (FIG. 19).

65. The absorbent article (2h) according to claim 63, characterized in that the center of the cover layer (39) is penetrated by an absorbent pad portion (51) extending in a longitudinal direction of the interlabial absorbent body, the one end (51a) of which forms the raised portion (40), and the opposite end (51b) of which reaches down between the material portions of the backing layer (10a) which are bent downwardly and extend opposite to each other (FIG. 20).

66. The absorbent article according to one of claims 55 to 58, characterized in that the interlabial absorbent body (2i) is formed as a flat pad (59), wherein, between cover layer (71) and backing layer (72), a layer (73) of cellulose fibers (14) which are at least partially present in the form of granules is arranged, wherein the main body of the pad (59) shows an oval outline, so that it resembles a fish body when seen from above, to which one end an attachment (61) is attached, which is disposed in the area of the pad and has a tail-fin shape (FIGS. 21 to 23).

67. The absorbent article according to claim 66, characterized in that the pad comprises a portion (60) drawn downwardly in the manner of a ventral fin, which imparts a T-like shape to the pad seen in a cross-section longitudinal to the plane XXII-XXII and serves as a handle for introducing the interlabial absorbent body (FIG. 22).

68. The absorbent article according to claim 67, characterized in that the portion drawn downwardly extends in a direction of the longitudinal axis of the interlabial absorbent body (2i) and/or in a way limited to the central longitudinal area.

69. The absorbent article according to one of claims 66 to 68, characterized in that the cover layer (71) is formed so as to be pleated (62) in a longitudinal direction (FIG. 22).
